SEQUENCE LISTING (19 sequences)

SEQ ID NO: 1 - protein, 8 aa, Artificial Sequence
  synthetic peptide
PAKRKLFG

SEQ ID NO: 2 - protein, 8 aa, Artificial Sequence
  synthetic peptide
PVKRRLDL

SEQ ID NO: 3 - protein, 8 aa, Artificial Sequence
  SYNTHETIC PROTEIN
SACRNLFG

SEQ ID NO: 4 - protein, 7 aa, Artificial Sequence
  SYNTHETIC PROTEIN
AKRKLFG

SEQ ID NO: 5 - protein, 6 aa, Artificial Sequence
  SYNTHETIC PROTEIN
KRKLFG

SEQ ID NO: 6 - protein, 5 aa, Artificial Sequence
  SYNTHETIC PROTEIN
RKLFG

SEQ ID NO: 7 - protein, 12 aa, Artificial Sequence
  SYNTHETIC PROTEIN
YGRKKRRQRRRG

SEQ ID NO: 8 - protein, 20 aa, Artificial Sequence
  SYNTHETIC PROTEIN
YGRKKRRQRRRGPVKRRLDL

SEQ ID NO: 9 - protein, 20 aa, Artificial Sequence
  SYNTHETIC PROTEIN
YGRKKRRQRRRGPAKRKLFG

SEQ ID NO: 10 - protein, 23 aa, Artificial Sequence
  SYNTHETIC PROTEIN
YGRKKRRQRRRGRLDLPKVRKRS

SEQ ID NO: 11 - protein, 23 aa, Artificial Sequence
  SYNTHETIC PROTEIN
YGRKKRRQRRRGETDHQYLAESS

SEQ ID NO: 12 - protein, 16 aa, Artificial Sequence
  SYNTHETIC PROTEIN
RQIKIWFQNRRMKWKK

SEQ ID NO: 13 - protein, 24 aa, Artificial Sequence
  SYNTHETIC PROTEIN
RQIKIWFQNRRMKWKKPVKRRLFG

SEQ ID NO: 14 - protein, 6 aa, Artificial Sequence
  SYNTHETIC PROTEIN
KKKLFG

SEQ ID NO: 15 - protein, 5 aa, Artificial Sequence
  Synthetic protein
KKLFG

SEQ ID NO: 16 - protein, 7 aa, Artificial Sequence
  synthetic protein
AKRKLFG

SEQ ID NO: 17 - protein, 6 aa, Artificial Sequence
  Synthetic protein
KRKLFG

SEQ ID NO: 18 - protein, 7 aa, Artificial Sequence
  Synthetic protein
AKKKLFG

SEQ ID NO: 19 - protein, 8 aa, Artificial Sequence
  Synthetic protein
PAKKKLFG

(19) United States
(12) Patent Application Publication
(10) Pub. No.: US 2004/0053849 A1
(43) Pub. Date: Mar. 18, 2004

(54) Inhibitors of the E2F-1/cyclin interaction for cancer therapy

(76) Inventors: Kenneth Walter Bair, Mountain Lakes, NJ (US); YingNan Pan Chen, Parsippany, NJ (US); Timothy Michael Ramsey, Sparta, NJ (US); Michael Lloyd Sabio, Randolph, NJ (US); Sushil Kumar Sharma, West Orange, NJ (US)

Correspondence Address: THOMAS HOXIE, NOVARTIS, CORPORATE INTELLECTUAL PROPERTY, ONE HEALTH PLAZA 430/2, EAST HANOVER, NJ, 07936-1080, US

(21) Appl. No.: 10/603,409

(22) Filed: Jun. 25, 2003

Related U.S. Application Data
(63) Continuation-in-part of application No. 10/024,935, filed on Dec. 19, 2001.
(60) Provisional application No. 60/256,828, filed on Dec. 20, 2000.

(51) Int. Cl. A61K 38/08; C07K 7/06

(52) U.S. Cl. 514/16; 514/17; 530/329; 530/330

(57) ABSTRACT

The novel compounds of this invention have the general structural formula Ia-d: The compounds of this invention relate to 8-mer, 7-mer, 6-mer and 5-mer peptides having the following amino acid sequences, and referred to collectively as having “Formula Ia-d”:

| | | |
|---|---|---|
| Cap-AA8-AA7-AA6-AA5-AA4*-AA3-AA2-AA1* | 8-mer | Ia |
| Cap-AA7-AA6-AA5-AA4*-AA3-AA2-AA1* | 7-mer | Ib |
| Cap-AA6-AA5-AA4*-AA3-AA2-AA1* | 6-mer | Ic |
| Cap-AA5-AA4*-AA3-AA2-AA1* | 5-mer | Id |

or a pharmaceutically acceptable salt or ester thereof, that inhibit the interaction of the transcription factor E2F-1 to Cyclin A. As an antagonist of the E2F-1/Cyclin A interaction, the compounds of the present invention may be used in the treatment of cancer.

### RELATIONSHIP TO OTHER APPLICATIONS

This application is a continuation-in-part of U.S. Ser. No. 10/024,935, filed Dec. 19, 2000, which was a non-provisional filing of Prov U.S. Ser. No. 60/256,828, filed Dec. 20, 2000; and a continuation-in part of U.S. Ser. No. ______, attorney docket 4-31664B filed Jun. 19, 2003, which was a 35 USC 371 filing of PCT/EP1/15006, filed Dec. 19, 2001, which in turn was a non-provisional filing of Prov U.S. Ser. No. 60/256,828, filed Dec. 20, 2000.

### FIELD OF THE INVENTION

The present invention relates generally to novel peptide compounds that inhibit the binding of the E2F-1 cell regulatory protein to Cyclin A. The present invention provides novel compounds, novel compositions, methods of their use and methods of their manufacture, where such compounds are generally pharmacologically useful as agents in therapies whose mechanism of action rely on the inhibition of the E2F-1/Cyclin A interaction, and more particularly useful in therapies for the treatment of cancer.

### BACKGROUND OF THE INVENTION

Recent studies have demonstrated a critical role for E2F-1 transcription activity on the regulation of cell growth, specifically during the G1/S phase transition. Rb family member proteins whose function is regulated by the G1 cyclin-dependent kinases (cdks) control the activity of the E2F family members. Disruption of various components of this control pathway is a regular event during the development of human cancer.

Progression through the mammalian cell cycle is driven by the orderly activation of cdks. Cdk activity is in turn regulated through post-translational modifications and by interaction with regulatory proteins such as cyclins. Each cyclin binds to a preferred subset of cdks and the resulting cyclin-cdk complexes typically display peak kinase activity for a defined period during the cell cycle.

One approach to treating cancer that minimizes host toxicity is to develop drugs that preferentially kill cells in which cell cycle pathways are altered. The use of in vitro kinase binding inhibition assays and in vivo growth suppression assays can identify compounds which are useful in treating cancers, or which can be further employed to provide scaffolds for the design of further novel peptidic and non-peptidic inhibitors.

In addition to its role in cell proliferation, several recent observations suggest the possibility that E2F-1 may be involved in apoptosis (programmed cell death). In particular, suppression of E2F-1 DNA-binding activity by Cyclin A/cdk2 is linked to orderly S phase progression; disruption of this linkage results in S phase delay and cell cycle arrest followed by apoptosis. Thus, disruption of the E2F-1/Cyclin A/cdk2 complex represents an attractive target for the development of antitumor drugs.

An ELISA was developed to identify antagonists of E2F-1/Cyclin A interaction. This method is based on interactions between three proteins, E2F-1, Cyclin A and cdk2 and is analyzed colorimetrically. This assay was used to determine IC₅₀ values for various synthetic peptides that were used in biological experiments and for SAR studies.

These synthetic peptides can be used as research tools to further investigate cell cycle regulation or as intermediates to make new conjugated (chimeric) peptides or further modified peptides and examined in cell growth inhibition assay. Peptides that cause cell growth inhibition and cell death in transformed cell lines can be used for cancer therapy in patients whose tumors-respond to-the-compounds, and used in-therapeutic regimens for cancer patients.

### SUMMARY OF THE INVENTION

The compounds of this invention are peptides comprising the amino acid sequence selected from the general structural formula Ia, Ib, Ic and Id:

| | | |
|---|---|---|
| Cap-AA8-AA7-AA6-AA5-AA4*-AA3-AA2-AA1* | 8-mer | Ia |
| Cap-AA7-AA6-AA5-AA4*-AA3-AA2-AA1* | 7-mer | Ib |
| Cap-AA6-AA5-AA4*-AA3-AA2-AA1* | 6-mer | Ic |
| Cap-AA5-AA4*-AA3-AA2-AA1* | 5-mer | Id |

or pharmaceutically acceptable salts thereof, that inhibit the interaction of the transcription factor E2F-1 to Cyclin A. As an antagonist of the E2F-1/Cyclin A interaction, the compounds of the present invention may be used in the treatment of cancer. There is no precedent in the literature for the inhibition of the E2F-1/Cyclin A interaction by cyclic peptides or non-peptides.

Therefore, it is an object of this invention to provide compounds that inhibit the E2F1/Cyclin A interaction. It is an additional object of this invention to provide methods of using the compounds of Formula Ia-d for the treatment of cancer. It is a further object of this invention to provide pharmaceutical compositions for the compounds of formula Ia-d. Still another object of the present invention is to provide a method for in vitro inhibition of the E2F-1/Cyclin A interaction using the compounds of formula Ia-d.

### DETAILED DESCRIPTION OF THE INVENTION

The present invention relates to isolated peptides comprising the amino acid sequence selected from the general structural formula Ia, Ib, Ic and Id:

| | | |
|---|---|---|
| Cap-AA8-AA7-AA6-AA5-AA4*-AA3-AA2-AA1* | 8-mer | Ia |
| Cap-AA7-AA6-AA5-AA4*-AA3-AA2-AA1* | 7-mer | Ib |
| Cap-AA6-AA5-AA4*-AA3-AA2-AA1* | 6-mer | Ic |
| Cap-AA5-AA4*-AA3-AA2-AA1* | 5-mer | Id |

wherein

AA1 is selected from:

(a) Glycine (Gly),

(b) Alanine (Ala),

(c) Leucine (Leu), and

(d) a small aliphatic amino acid;

AA2 is selected from:

(a) Phenylalanine (Phe),

(b) Thienylalanine (Tha),

(c) Cyclohexylalanine (Cha),

(d) Tyrosine (Tyr),

(e) Pyridylalanine (Pya),

(f) Tryptophan (Trp), and

(g) another aromatic amino acid;

AA3 is selected from:

(a) Leu,

(b) Cyclopropylalanine (Cpa), and

(c) a natural or unnatural aliphatic amino acid;

AA4 is selected from:

(a) Lysine (Lys),

(b) Lys substituted by C₁-C₁₇ alkyl, or C₁-C₁₇ alkyl substituted with cyclized saturated or unsaturated C₅-C₂₀ alkyl; C₅-C₂₀ arylalkyl or a C₆-C₂₀ aryl radical, or Lys wherein N^{ε} is substituted by one or two radicals selected from C₅-C₂₀ alkyl; a C₁-C₁₇ alkyl substituted with cyclized saturated or unsaturated C₅-C₂₀ alkyl; or mono or di-hetero alkyl, such as piperazinyl; the latter can be further substituted by a C₁-C₁₇ alkyl substituted with cyclized saturated or unsaturated C₅-C₂₀ alkyl; or the N^{ε} can be part of a ring with an optional additional N atom, such as piperazinyl, the ring being optionally further substituted with C₁-C₁₇ alkyl substituted with cyclized saturated or unsaturated C₅-C₂₀ alkyl; or N^{ε} is substituted with a linear or branched C₁-C₆ acyl group; a cyclized saturated or unsaturated C₅-C₂₀ alkyl; a C₅-C₂₀ arylalkyl such as benzyl or a C₆-C₂₀ aryl radical such as phenyl;

(c) Ornithine (Orn) optionally substituted by C₁-C₁₇ alkyl, C₅-C₂₀ arylalkyl or a C₆-C₂₀ aryl radical, and

(d) Homolysine (hLys) optionally substituted by C₁-C₁₇ alkyl, C₅-C₂₀ arylalkyl or a C₆-C₂₀ aryl radical;

AA5 is selected from:

(a) Arginine (Arg),

(b) Lys,

(c) Orn,

(d) hLys,

(e) Histidine (His), and

(f) Lys substituted by C₁-C₁₇alkyl, or C₁-C₁₇alkyl substituted with cyclized saturated or unsaturated C₅-C₂₀ alkyl; C₅-C₂₀ arylalkyl or a C₆-C₂₀ aryl radical, or Lys wherein N^{ε} is substituted by one or two radicals selected from C₅-C₂₀ alkyl; a C₁-C₁₇ alkyl substituted with cyclized saturated or unsaturated C₅-C₂₀ alkyl; or mono or di-hetero alkyl, such as piperazinyl; the latter can be further substituted by a C₁-C₁₇ alkyl substituted with cyclized saturated or unsaturated C₅-C₂₀ alkyl; or the N^{ε} can be part of a ring with an optional additional N atom, such as piperazinyl, the ring being optionally further substituted with C₁-C₁₇ alkyl substituted with cyclized saturated or unsaturated C₅-C₂₀ alkyl; or N^{ε} is substituted with a linear or branched C₁-C₆ acyl group; a cyclized saturated or unsaturated C₅-C₂₀ alkyl; a C₅-C₂₀ arylalkyl such as benzyl or a C₆-C₂₀ aryl radical such as phenyl;

AA6 is selected from:

(a) Lys,

(b) hLys,

(c) Orn,

(d) Lys substituted by C₁-C₁₇ alkyl, or C₁-C₁₇ alkyl substituted with cyclized saturated or unsaturated C₅-C₂₀ alkyl; C₅-C₂₀ arylalkyl or a C₈-C₂₀ aryl radical, or Lys wherein N^{ε} is substituted by one or two radicals selected from C₅-C₂₀ alkyl; a C₁-C₁₇ alkyl substituted with cyclized saturated or unsaturated C₅-C₂₀ alkyl; or mono or di-hetero alkyl, such as piperazinyl; the latter can be further substituted by a C₁-C₁₇ alkyl substituted with cyclized saturated or unsaturated C₅-C₂₀ alkyl; or the N^{ε} can be part of a ring with an optional additional N atom, such as piperazinyl, the ring being optionally further substituted with C₁-C₁₇ alkyl substituted with cyclized saturated or unsaturated C₅-C₂₀ alkyl; or N^{ε} is substituted with a linear or branched C₁-C₆ acyl group; a cyclized saturated or unsaturated C₅-C₂₀ alkyl; a C₅-C₂₀ arylalkyl such as benzyl or a C₆-C₂₀ aryl radical such as phenyl;

(e) Orn wherein N^{δ} is substituted by one or two radicals selected from C₅-C₂₀ alkyl, a linear or branched C₁-C₆ acyl group, cyclized saturated or unsaturated C₅-C₂₀ alkyl, C₅-C₂₀ arylalkyl such as benzyl, and a C₆-C₂₀ aryl radical such as phenyl, and

(f) Proline (Pro).

AA7 is selected from:

(a) Ala,

(b) Valine (Val), and

(c) a natural or unnatural amino acid, or mimetics or isostere thereof;

AA8 is selected from:

(a) Proline (Pro),

(b) a natural or unnatural amino acid, or mimetics or isostere thereof; and

the Cap is either not present or preferably selected from but not limited to:

(a) C₁-C₈ acyl, and

(b) C₃-C₈ cycloalkylalkanoyl or furanylacetyl;

and pharmaceutically acceptable salts thereof; such peptides being preferably linked to nuclear localization peptide sequences such as, but not limited to, HIV-1 Tat or Antennapedia peptide sequence (penetratin). The (*) symbol indicates a site for intramolecular linkage. The intramolecular linkage is via an amide, substituted amide bond or isostere thereof. When any of the peptides above are linked through the starred (*) amino acids, the compounds are cyclic 5-mers, 6-mers, 7-mers, or 8-mers. The cyclic mers are preferred over the linear mers. These peptides can also be polyamino acid fragments that are connected to other amino acids as desired. In the same manner, the N-terminal of each peptide-sequence-can-be capped-by-a “Cap” group. Any amino-acid can be replaced by their mimetics, isosteres or analogs.

Preferably, the present invention relates to isolated peptides comprising the amino acid sequence selected from the general structural formula Ia, Ib, Ic and Id:

| | | |
|---|---|---|
| Cap-AA8-AA7-AA6-AA5-AA4*-AA3-AA2-AA1* | 8-mer | Ia |
| Cap-AA7-AA6-AA5-AA4*-AA3-AA2-AA1* | 7-mer | Ib |
| Cap-AA6-AA5-AA4*-AA3-AA2-AA1* | 6-mer | Ic |
| Cap-AA5-AA4*-AA3-AA2-AA1* | 5-mer | Id |

wherein

AA1 is selected from:

(a) Gly,

(b) Ala, and

(c) Leu;

AA2 is selected from:

(a) Phe,

(b) Tha,

(c) Cha,

(d) Tyr,

(e) Pya, and

(f) Trp;

AA3 is selected from:

(a) Leu,

(b) Cpa, and

(c) a natural aliphatic amino acid;

AA4 is selected from:

(a) Lys,

(b) Orn, and

(c) hLys;

AA5 is selected from:

(a) Arg,

(b) Lys,

(c) Orn,

(d) hLys, and

(e) His;

AA6 is selected from:

(a) Lys,

(b) hLys,

(c) Orn;

AA7 is selected from:

(a) Ala,

(b) Val, and

(c) a natural amino acid;

AA8 is selected from:

(a) Pro,

(b) a natural amino acid; and

the Cap is either not present or preferably selected from:

(a) acetyl (Ac), cyclopropylcarbonyl, cyclopropylacetyl (Cpr), pivaloyl, isopropylcarbonyl, isopropylacetyl, 2,2-dimethylbutanoyl (Dmb), levulinoyl, cyclopropylglycinoyl (Cpg), dimethylglycinoyl (Dmg), and

(b) cyclopentylacetyl, cyclohexylacetyl, cycloheptylacetyl, furanylacetyl;

and pharmaceutically acceptable salts thereof;

such peptides being optionally linked to nuclear localization peptide sequences HIV-1 Tat or Antennapedia peptide sequence (penetratin);

and the (*) symbol indicates a site for optional intramolecular linkage via an amide bond; the resulting compounds being the respective cyclic 5-mers, 6-mers, 7-mers, or 8-mers.

Preferred examples of compounds within this class include, but are not limited to, the following:

Cyclic 5-mer:

Ac-Arg-(Lys-Leu-Phe-Gly), or

Ac-Lys-(Lys-Leu-Phe-Gly);

Cyclic 6-mer:

Ac-Lys-Arg-(Lys-Leu-Phe-Gly),

Ac-Lys-Lys-(Lys-Leu-Phe-Gly),

Cpr-Lys-Arg-(Lys-Leu-Phe-Gly),

Cpr-Lys-Lys-(Lys-Leu-Phe-Gly),

Cpr-Lys-(C₅-C₂₀)-Lys-(Lys-Leu-Phe-Gly),

Cpr-Lys-(C₅-C₂₀)-Arg-(Lys-Leu-Phe-Gly),

Cpr-Lys-(CH(CH₃)(C₁₃H₂₇))-Lys-(Lys-Leu-Phe-Gly), [see Example 1]

Dmb-Lys-(C₅-C₂₀)-Arg-(Lys-Leu-Phe-Gly), or

Dmb-Lys-(C₅-C₂₀)-Lys-(Lys-Leu-Phe-Gly);

Cyclic 7-mer:

Ac-Ala-Lys-Arg-(Lys-Leu-Phe-Gly),

Ac-Ala-Lys-Lys-(Lys-Leu-Phe-Gly),

Cpr-Ala-Lys-Arg-(Lys-Leu-Phe-Gly), or

Cpr-Ala-Lys-Lys-(Lys-Leu-Phe-Gly);

Cyclic 8-mer:

Pro-Ala-Lys-Arg-(Lys-Leu-Phe-Gly), [see Example 2]

Ac-Pro-Ala-Lys-Arg-(Lys-Leu-Phe-Gly),

Ac-Pro-Ala-Lys-Lys-(Lys-Leu-Phe-Gly),

Cpr-Pro-Ala-Lys-Arg-(Lys-Leu-Phe-Gly), or

Cpr-Pro-Ala-Lys-Lys-(Lys-Leu-Phe-Gly);

wherein parentheses indicate the residues involved in cyclization;

and pharmaceutically acceptable salts thereof.

The compounds of the present invention are named by reference to an octapeptide of the general format:

Cap-AA8-AA7-AA6-AA5-AA4-AA3-AA2-AA1

where “AAX” represents the amino acid in the “xth” (x=1-8) position in the octapeptide starting from AA1 at the C-terminus. The ‘Cap’ is a non-amino acid group attached to the N-terminus. AA1 is the carboxy terminal residue. Names are given in the general form: amino terminus ‘cap’, followed by the three letter code of the first residue, followed by a hyphen and the three letter code of the second residue, followed by a hyphen and the three letter code of the third residue, and so on (three letter code is standard peptide nomenclature: see Amino Acid and Peptide Nomenclature J. Biol. Chem 260, 14-42 and IUPAC-IUB Nomenclature recommendations). Unnatural amino acids are referred to by accepted nomenclature.

The term “peptide” as used herein is understood to include also polypeptides where appropriate.

As used herein “alkyl” is intended to include both branched- and straight-chain saturated or unsaturated aliphatic hydrocarbon groups having the specified number of carbon atoms. “Acyl” represents an alkyl group having the indicated number of carbon atoms attached through a —C(O)— bridge.

As used herein, the term “isolated” means that the material is removed from its original environment, e.g. the natural environment if it is naturally occurring.

The compounds of this invention are linear and cyclic analogues of the sequence: Ac-Pro-Ala-Lys-Arg-Lys-Leu-Phe-Gly. The linear sequence is a consensus sequence of several cell cycle regulatory proteins that bind to Cyclin A, effectively inhibiting the binding of the E2F-1 to Cyclin A. A number of compounds which provide the desired level of inhibitory activity were identified.

The original identified sequences are shown in Table I:

TABLE I
Original Identified Sequences

| Sequence | Source | IC₅₀ (nM) |
|---|---|---|
| Pro-Val-Lys-Arg-Arg-Leu-Asp-Leu | From E2F-1 | 10 |
| Pro-Ala-Lys-Arg-Lys-Leu-Phe-Gly | Consensus Sequence | 100 |
| Ser-Ala-Cys-Arg-Asn-Leu-Phe-Gly | p27 Sequence | 200 |

Of these, the minimum peptide length required for inhibition of the E2F-1/Cyclin A interaction was determined and is shown in Table II:

TABLE II
IC₅₀ of Linear and Cyclic Analogs

IC₅₀ (nM)

| Peptide | Linear | Cyclic |
|---|---|---|
| Pro-Ala-Lys-Arg-Lys-*Leu-Phe-Gly* | 100 | 1 |
| Ac-Ala-Lys-Arg-Lys-*Leu-Phe-Gly* | 200 | 10 |
| Ac-Lys-Arg-Lys-*Leu-Phe-Gly* | 1,000 | 20 |
| Ac-Arg-Lys-*Leu-Phe-Gly* | 30,000 | 3,000 |

The peptides are cyclized between the side-chain amino group of Lys* and the carboxyl group of Gly*. Note: The sequence Pro-Ala-Lys-Arg-Lys-*Leu-Phe-Gly* has been represented here onwards as Pro-Ala-Lys-Arg-(Lys-Leu-Phe-Gly) as per accepted general convention indicating that residues in the bracket are involved in a cycle. The minimum sequence required for effective inhibition is 5-8 residues. The cyclic analogs are generally 50-100 fold better inhibitors than the corresponding linear analogs.

The critical residues in the consensus sequence, Pro⁸-Ala⁷-Lys⁶-Arg⁵-Lys⁴-Leu³-Phe²-Gly¹ required for the inhibition of the E2F-1/Cyclin A interaction are Lys⁶, Arg⁵, Leu³, and Phe². The optimally active peptide is the cyclized consensus sequence, however, Pro⁸, Ala⁷ and/or Lys⁶ can be replaced with other amino acids, mimetics, isosteres or analogs. In the case of Lys⁶, it can be replaced with other amines and thiols such as cysteine (Cys), 5-aminovaleric acid, 6-aminocaproic acid, and levulinic acid. It can also be replaced by hLys, Orn, or Lys with N^{ε} and Orn with N^{δ} substituents, which are C₅-C₂₀ linear or branched, straight chain or cyclized saturated or unsaturated alkyl, or can be replaced by a C₆-C₂₀ aryl radical such as phenyl, C₅-C₂₀ arylalkyl such as benzyl, or Arg. Ala⁷ can be replaced by Pro, a linear or branched acyl group.

In the case of Arg⁵, replacement is detrimental to activity, although the peptides can accept Arg mimetics, isostere or analogs. Leu³ is critical for activity. The presence of Phe² is also critical, although it may accept other mimetics, isosteres or analogs, preferably an aromatic or hydrophobic group. Gly¹ is required for cyclic peptides.

Identification of Critical Residues in Cyclic 8-mer, Pro-Ala-Lys-Arg-(Lys-Leu-Phe-Gly)

In order to evaluate the role of the individual amino acids of cyclic 8-mer, Pro-Ala-Lys-Arg-(Lys-Leu-Phe-Gly) in binding to Cyclin A, each amino acid was replaced with an isostere and the inhibitory activity was measured in an in vitro ELISA.

A. Replacement of Phenylalanine (Phe, F)

Several analogs of cyclic 8-mer, Pro-Ala-Lys-Arg-(Lys-Leu-Phe-Gly) were synthesized using various Phe isosteres and their activity determined.

The data indicates that all the amino acid isosteres used for Phe did not result in an increase in the activity of the original lead cyclic 8-mer, Pro-Ala-Lys-Arg-(Lys-Leu-Phe-Gly). However, unnatural amino acids such as Tha and Cha can replace Phe without much loss in activity of the cyclic 8-mer, Pro-Ala-Lys-Arg-(Lys-Leu-Phe-Gly).

B. Replacement of Leucine (Leu, L)

Several analogs of cyclic 8-mer, Pro-Ala-Lys-Arg-(Lys-Leu-Phe-Gly) were synthesized using various Leu isosteres and their activity determined. The results suggest that of all the amino acid isosteres used to replace Leu did not result in an increase in the activity of the original lead 2. However, unnatural amino acids such as Cpa can replace Leu with no loss in activity.

C. Identification of Minimum Sequence Required for Inhibition

Cyclic peptides were synthesized by sequential removal of amino acids from the N-terminal of the cyclic 8-mer peptide, Pro-Ala-Lys-Arg-(Lys-Leu-Phe-Gly) followed by capping with an acetyl group and these peptides were analyzed for their inhibitory activities in an in vitro ELISA (Table III). The absolute minimum sequence required for the E2F-1/Cyclin A interaction is a cyclic 6-mer, 4.

TABLE III
IC₅₀ of Various Peptides

| Peptide Sequence | ELISA IC₅₀ (nM) |
|---|---|
| Pro-Ala-Lys-Arg-(Lys-Leu-Phe-Gly),2 | 1 |
| Ac-Ala-Lys-Arg-(Lys-Leu-Phe-Gly),3 | 10 |
| Ac-Lys-Arg-(Lys-Leu-Phe-Gly),4 | 20 |
| Ac-Arg-(Lys-Leu-Phe-Gly),5 | 1,000 |

The acetyl group of 4 was replaced by several other acyl groups and the activity of each analog was measured in an ELISA. The replacement of the acetyl group with an isopropylcarbonyl, isopropylacetyl-, pivaloyl-, and cyclopropylcarbonyl, cyclopropylacetyl-group provided 6-mer analogs that are equipotent to the cyclic 8-mer, Pro-Ala-Lys-Arg-(Lys-Leu-Phe-Gly). This manipulation of a secondary hydrophobic pocket residue allows removal of two amino acids from the cyclic 8-mer, Pro-Ala-Lys-Arg-(Lys-Leu-Phe-Gly) without losing inhibitory activity.

The compounds of this invention may be prepared from their constituent amino acids using standard methods of protein synthesis, e.g., Schroeder et al., “The Peptides”, Vol. I, Academic Press, 1965, or Bodanszky et al., “Peptide Synthesis”, Interscience Publishers 1966, or McOmie (ed.), “Protective Groups in Organic Chemistry”, Plenum Press 1973, and “The Peptides. Analysis, Synthesis, Biology” 2, Chapter I by George Barany and R. B. Merrifield, Academic Press, 1980, New York.

The condensation of two amino acids, or an amino acid and a peptide, or two peptides can be carried out according to the usual condensation methods such as azide method, mixed acid anhydride method, carbodiimide method, active ester method (p-nitrophenyl ester method, BOP [benzotriazol-1-yloxy-tris-(dimethylamino)-phosphonium hexafluorophosphate] method, N-hydroxysuccinic acid imido ester method, etc.), Woodward reagent K method, or HBTU method. In the case of elongating the peptide chain in the solid phase method, the peptide is attached to an insoluble carrier at the C terminal amino acid. For insoluble carriers, those which react with the carboxyl group of the C-terminal amino acid to form a bond which is readily cleaved later, e.g., a halomethyl resin such as chloromethyl resin and bromomethyl resin, hydroxymethyl resin, aminomethyl resin, p-hydroxymethylphenyl acetamide (PAM) resin, benzhydrylamine resin, t-alkyloxycarbonyl-hydrazide resin, or sasrin, Wang or trityl resins can be used.

Common to chemical syntheses of peptides is the protection of the reactive side-chain groups of the various amino acid moieties with suitable protecting groups at that site until the group is ultimately removed after the chain has been completely assembled. Also common is the protection of the alpha-amino group on an amino acid or a fragment while that entity reacts at the carboxyl group followed by the selective removal of the alpha-amino-protecting group to allow subsequent reaction to take place at that location. Accordingly, it is common that as a step in the synthesis, an intermediate compound is produced which includes each of the amino acid residues located in the desired sequence in the peptide chain with various of these residues having side-chain protecting groups. These protecting groups are then commonly removed substantially at the same time so as to produce the desired resultant product following purification.

The applicable protective groups for protecting the alpha-and omega-side-chain amino groups are, e.g., benzyloxycarbonyl, isonicotinyloxycarbonyl-(iNOC), o-chlorobenzyloxycarbonyl, p-nitrobenzyloxycarbonyl, p-methoxybenzyoxycarbonyl, t-butoxycarbonyl (Boc), t-amyloxycarbonyl (Aoc), isobornyloxycarbonyl, adamantyloxycarbonyl, 2(4,4-biphenyl)-2-propyloxycarbonyl (Bpoc), 9-fluorenylmethoxycarbonyl (Fmoc), methylsulfonylethoxycarbonyl (Msc), trifluoroacetyl, phthalyl, formyl, 2-nitrophenylsulphenyl (NPS), diphenylphosphinothioyl (Ppt), dimethylphosphinothioyl (Mpt), and the like.

As protective groups for the carboxyl group there can be exemplified, for example, benzyl ester (Bzl), t-butyl cycloester (t-Bu), 4-pyridylmethyl ester (OPic), and the like. It is desirable that specific amino acids such as Arg, Cys, and serine (Ser) possessing a functional group other than amino and carboxyl groups are protected by a suitable protective group as occasion demands. For example, the guanidino group in Arg may be protected with nitro, p-toluenesulfonyl, benzyloxycarbonyl, adamantyloxycarbonyl, p-methoxybenzenesulfonyl, 4-methoxy-2,6-dimethylbenzenesulfonyl (Mds), 1,3, 5-trimethylphenysulfony/l (Mts), 2,2,4,6,7-pentamethyldihydrobenzofuran-5-sulfonyl (PBF), trityl, and the like. The thiol group in cysteine may be protected with p-methoxybenzyl, triphenylmethyl, acetylaminomethyl, ethylcarbamoyl, 4-methylbenzyl, 2,4,6-trimethybenzyl (Tmb) etc., and the hydroxyl group in Ser can be protected with benzyl, t-butyl, acetyl, tetrahydropyranyl etc.

Stewart and Young in “Solid Phase Peptide Synthesis”, Pierce Chemical Company, Rockford, Ill. (1984), provide detailed information regarding procedures for preparing peptides. Protection of alpha-amino groups is described on pages 14-18, and side-chain blockage is described on pages 18-28. A table of protecting groups for amine, hydroxyl and sulfhydryl functions is provided on pages 149-151. These descriptions are hereby incorporated by reference.

The peptides of the present invention also may be prepared using manufacturer supplied protocols with automated peptide synthesizing machines, e.g., Beckman, Applied Biosystems Inc., or Milligen Co. An Applied Biosystems ABI 433A peptide synthesizer using standard Fmoc protocol was used. The desired amino acid derivatives and resins were purchased from commercial sources. Reverse-phase HPLC was carried out with a commercial HPLC system on YMC C18 columns using a linear gradient of acetonitrile/0.1% aqueous TFA. The elution was monitored at 215, 230, 254, and 280 nm. The purified peptides were analyzed by mass spectrometric techniques. Peptides were labeled with fluorescein using fluorescein-5-maleimide and DIEA-(4 eq.) in DMF on their Cys residue.

The compounds of the present invention can be prepared readily according to the following Examples or modifications thereof using readily available starting materials, reagents and conventional synthesis procedures. In these reactions, it is also possible to make use of variants, which are themselves known to those of ordinary skill in this art, but are not mentioned in greater detail.

In an embodiment, the present invention provides a method of inhibiting the binding of the E2F-1 cell regulatory protein to Cyclin A comprising administering to a mammal in need of such treatment a therapeutically effective amount of a peptide of the invention, or a pharmaceutically acceptable salt thereof.

The ability of the peptides of the present invention, and their corresponding pharmaceutically acceptable salts, to inhibit the binding of the E2F-1 cell regulatory protein to Cyclin A may be demonstrated employing an ELISA based on interactions between three proteins, E2F-1, Cyclin A and cdk2. This assay allows determination of IC₅₀ values for various synthetic peptides that were used in biological experiments or for SAR studies.

In a further embodiment, the present invention provides a method for treating cancer comprising administering to a mammal in need of such treatment a therapeutically effective amount of a peptide of the invention, or a pharmaceutically acceptable salt thereof.

The present invention also includes pharmaceutical compositions useful in inhibiting the binding of the E2F-1 cell regulatory protein to Cyclin A comprising a pharmaceutically acceptable carrier or diluent and a therapeutically effective amount of a peptide of the invention, or a pharmaceutically acceptable salt thereof.

The present invention further provides a peptide of the invention, or a pharmaceutically acceptable salt thereof, for use in a method for the therapeutic treatment of a mammal.

In another embodiment, the present invention provides a pharmaceutical composition comprising a peptide of the invention, or a pharmaceutically acceptable salt thereof, together with a pharmaceutically acceptable carrier.

In yet another embodiment, the present invention provides a pharmaceutical composition for the treatment of cancer in a mammal comprising, in a therapeutically effective amount, a peptide of the invention, or a pharmaceutically acceptable salt thereof, together with a pharmaceutically acceptable carrier.

The present invention also relates to the use of a peptide of the invention, or a pharmaceutically acceptable salt thereof, for the preparation of a pharmaceutical composition for use in the treatment of cancer.

The present invention further also relates to the use of a peptide of the invention, or a pharmaceutically acceptable salt thereof, in the treatment of cancer.

The compounds of the present invention may be administered in the form of pharmaceutically acceptable salts. The term “pharmaceutically acceptable salt” is intended to include all acceptable salts such as acetate, lactobionate, benzenesulfonate, laurate, benzoate, realate, bicarbonate, maleate, bisulfate, mandelate, bitartrate, mesylate, borate, bromide, methylnitrate, calcium edetate, methylsulfate, camsylate, mucate, carbonate, napsylate, chloride, nitrate, clavulanate, N-methylglucamine, citrate, ammonium salt, dihydrochloride, oleate, edetate, oxalate, edisylate, pamoate (embonate), estolate, palmitate, esylate, pantothenate, fumarate, phosphate/diphosphate, gluceptate, polygalacturonate, gluconate, salicylate, glutamate, stearate, glycolylarsanilate, sulfate, hexylresorcinate, subacetate, hydrabamine, succinate, hydrobromide, tannate, hydrochloride, tartrate, hydroxynaphthoate, teoclate, iodide, tosylate, isothionate, lactate, panoate, valerate, and the like which can be used as a dosage form for modifying the solubility or hydrolysis characteristics or can be used in sustained release or pro-drug formulations. Depending on the particular functionality of the compound of the present invention, pharmaceutically acceptable salts of the compounds of this invention include those formed from cations such as sodium, potassium, aluminum, calcium, lithium, magnesium, zinc, and from bases such as ammonia, ethylenediamine, N-methyl-glutamine, lysine, arginine, omithine, choline, N,N′-dibenzylethylenediamine, chloroprocaine, diethanolamine, procaine, N-benzylphenethylamine, diethylamine, piperazine, tris-(hydroxymethyl)aminomethane, and tetramethylammonium hydroxide.

These salts may be prepared by standard procedures, e.g., by reacting a free acid with a suitable organic or inorganic base. Where a basic group is present, such as amino, an acidic salt, i.e., hydrochloride, hydrobromide, trifluoroacetate, acetate, pamoate, and the like, can be used as the dosage form.

Also, in the case of an acid (—COOH) or alcohol group being present, pharmaceutically acceptable esters can be employed, e.g., acetate, maleate, pivaloyloxymethyl, and the like, and those esters known in the art for modifying solubility or hydrolysis characteristics for use as sustained release or prodrug formulations.

The compounds of the present invention or derivatives thereof may have chiral centers other than those centers whose stereochemistry is depicted in Formula Ia-d, and therefore may occur as racemates, racemic mixtures and as individual enantiomers or diastereomers, with all such isomeric forms being included in the present invention as well as mixtures thereof. Furthermore, some of the crystalline forms for compounds of the present invention or derivatives thereof may exist as polymorphs and as such are intended to be included in the present invention. In addition, some of the compounds of the instant invention may form solvates with water or common organic solvents. Such solvates are encompassed within the scope of this invention.

The term “therapeutically effective amount” means that amount of a drug or pharmaceutical agent that will elicit the biological or medical response of a tissue, system, animal or human that is being sought by a researcher, veterinarian, medical doctor or other clinician, which includes alleviation of the symptoms of the disorder being treated. The novel methods of treatment of this invention are for disorders known to those skilled in the art.

The term “mammal” includes humans.

The dosage regimen utilizing the compounds of the present invention is selected in accordance with a variety of factors including type, species, age, weight, sex and medical condition of the patient; the severity of the condition to be treated; the route of administration; the renal and hepatic function of the patient; and the particular compound thereof employed. A physician or veterinarian of ordinary skill can readily determine and prescribe the effective amount of the drug required to prevent, counter or arrest the progress of the condition. Optimal precision in achieving concentration of drug within the range that yields efficacy without toxicity requires a regimen based on the kinetics of the drug's availability to target sites. This involves a consideration of the distribution, equilibrium, and elimination of a drug.

The daily dosage of the products may be varied over a range from 0.01 to 500 mg per adult human per day. For oral administration, the compositions are preferably provided in the form of tablets containing from 0.01 to 500 mg, preferably 0.01, 0.05, 0.1, 0.5, 1.0, 2.5, 5.0, 10.0, 15.0, 25.0, or 50.0 mg of the active ingredient for the symptomatic adjustment of the dosage to the patient to be treated. An effective amount of the drug is ordinarily supplied at a dosage level of from about 0.001 mg/kg to about 50 mg/kg of body weight per day. The range is more particularly from about 0.01 mg/kg to 10 mg/kg of body weight per day.

For the treatment of cancer, the compounds of the present invention may be used together with agents known to be useful in treating cancer.

For combination treatment with more than one active agent, where the active agents are in separate dosage formulations, the active agents can be administered concurrently, or they each can be administered at separately staggered times.

The compounds of this invention can be delivered orally, intravenously, intrathecally, or parentally, in carriers or linked to chaperone carriers to effect delivery to the target site in the body.

The present invention also has the objective of providing suitable oral, systemic and parenteral pharmaceutical formulations for use in the novel methods of treatment of the present invention. The term “treatment” is intended to include ameliorating symptoms and/or arresting the progression of cancer in an individual known to be, or believed to be suffering from cancer. The term “administration of” or “administering a” compound should be understood to mean providing a compound of the invention or a prodrug of a compound of the invention to the individual in need of treatment. The compositions containing the present compounds as the active ingredient for use in the treatment of the above-noted conditions can be administered in a wide variety of therapeutic dosage forms in conventional vehicles for systemic administration. For example, the compounds can be administered in such oral dosage forms as tablets, capsules (each including timed release and sustained release formulations), pills, powders, granules, elixirs, tinctures, solutions, suspensions, syrups and emulsions, or by injection. Likewise, they may also be administered in intravenous (both bolus and infusion), intraperitoneal, intrathecally, subcutaneous, topical with or without occlusion, or intramuscular form, all using forms well known to those of ordinary skill in the pharmaceutical arts.

In the methods of the present invention, the compounds herein described in detail can form the active ingredient, and are typically administered in admixture with suitable pharmaceutical diluents or excipients suitably selected with respect to the intended form of administration, that is, oral tablets, capsules, elixirs, syrups and the like, and consistent with conventional pharmaceutical practices.

For instance, for oral administration in the form of a tablet or capsule, the active drug component can be combined with an oral, non-toxic pharmaceutically acceptable inert carrier such as ethanol, glycerol, water and the like. Moreover, when desired or necessary, suitable binders, lubricants, disintegrating agents and coloring agents can also be incorporated into the mixture. Suitable binders include, without limitation, starch, gelatin, natural sugars such as glucose or beta-lactose, corn sweeteners, natural and synthetic gums such as acacia, tragacanth or sodium alginate, carboxymethylcellulose, polyethylene glycol, waxes and the like. Lubricants used in these dosage forms include, without limitation, sodium oleate, sodium stearate, magnesium stearate, sodium benzoate, sodium acetate, sodium chloride and the like. Disintegrators include, without limitation, starch, methyl cellulose, agar, bentonite, xanthan gum and the like.

The liquid forms in suitably flavored suspending or dispersing agents such as the synthetic and natural gums, for example, tragacanth, acacia, methyl-cellulose and the like. Other dispersing agents which may be employed include glycerin and the like. For parenteral administration, sterile suspensions and solutions are desired. Isotonic preparations which generally contain suitable preservatives are employed when intravenous administration is desired.

The compounds of the present invention can also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

The compounds of the present invention may be coupled to a class of biodegradable polymers useful in achieving controlled release of a drug, for example, polylactic acid, polyepsilon caprolactone, polyhydroxy butyric acid, polyorthoesters, polyacetals, polydihydropyrans, polycyanoacrylates and cross-linked or amphipathic block copolymers of hydrogels.

### GENERAL METHODS

All temperatures given in the following examples are in degrees Celsius. Except where indicated, commercially available compounds were used without further purification. Where not noted, natural and unnatural amino acids are of the (L) configuration.

Peptide Synthesis

Peptides are assembled on an Applied Biosystems ABI 433A peptide synthesizer using standard Fmoc protocol. Amino acid derivatives and resins are purchased from Bachem Bioscience and Midwest Biotech. Reverse-phase HPLC is carried out with Waters HPLC systems on YMC C18 columns using linear gradients of acetonitrile/0.1% aqueous TFA. The elution is monitored at 215, 230, 254, and 280 nm. The purified peptides are analyzed by mass spectrometry (SCIEX API III mass spectrometer).

Examples of generally accepted abbreviations employed are shown in Tables IV and V:

TABLE IV
Abbreviations Used in Text

| | |
|---|---|
| ABTS | 2,2′-Azino-bis-(3-ethylbenzthiazoline-sulfonic acid) |
| Ala | Alanine |
| Arg | Arginine |
| BSA | Bovine serum albumin |
| Cdk | Cyclin-dependent kinase |
| Cha | Cyclohexylalanine |
| Cpa | Cyclopropylalanine |
| Cpr | Cyclopropylacetyl |
| Dmb | 2,2-dimethylbutyric acid |
| DMEM | 4′,6-Diamidino-2-phenylindole hydrochloride |
| ELISA | Enzyme-linked immunosorbant assay |
| FBS | Fetal calf serum |
| Gly | Glycine |
| HEPES | N-[2-Hydroxyethyl]piperazine-N′-[2-ethanesulfonic acid] |
| h-Lys | Homolysine |
| HOBt | 1-Hydroxybenzotriazole |
| HPLC | High pressure liquid chromatography |
| HRP | Horse radish peroxidase |
| IC₅₀ | 50% inhibitory concentration |
| Leu | Leucine |
| Lys | Lysine |
| Mtt | Methyltrityl |
| Orn | Ornithine |
| PBF | 2,2,4,6,7-pentamethyldihydrobenzofuran-5-sulfonyl |
| Phe | Phenylalanine |
| Phg | Phenylglycine |
| Pro | Proline |
| Pya | Pyridylalanine |
| SAR | Structure-activity relationship |
| TBS | Tris buffered saline |
| TBST | Tris buffered saline + 0.1% Tween 20 |
| Tha | Thienylalanine |
| Val | Valine |

TABLE V
Universal Single Amino Acid Codes

| CODE | Amino Acid |
|---|---|
| A | Alanine |
| C | Cysteine |
| D | Aspartic acid |
| E | Glutamic acid |
| F | Phenylalanine |
| G | Glycine |
| H | Histidine |
| I | Isoleucine |
| K | Lysine |
| L | Leucine |
| M | Methionine |
| N | Asparagine |
| P | Proline |
| Q | Glutamine |
| R | Arginine |
| S | Serine |
| T | Threonine |
| V | Valine |
| W | Tryptophan |
| Y | Tyrosine |

Biological Assays

Materials and Methods to Determine Growth Inhibition in Tumor Cells by Specific Peptides

Cell Lines (Table VI). MDA-MB-435, U2OS, A549, MDA-MB-231 cells were maintained in DMEM supplemented with 10% FCS. SW480 and HCT-116 were grown in RPMI 1640 supplemented with 10% FCS.

TABLE VI
Human Cell Lines Used in the Assay

| Cell line Abbreviation | Cell type* |
|---|---|
| MDA-MB-435 | breast carcinoma |
| MDA-MB-231 | breast carcinoma |
| U2OS | colon carcinoma |
| A549 | lung carcinoma |
| SW480 | colon carcinoma |
| HCT-116 | colon carcinoma |
| WI38/VA13 SV40 | SV40 transformed human lung fibroblast |

*All cell lines were aquired from American Type Culture Collection, Rockville, MD.

Peptide Treatment and Fluorescence Microscopy. 4×10⁴ cells/well were plated on a 48 well plate at 10% FCS and cultured overnight. The culture medium was discarded, and the cells were washed once with Opti-MEM. The cell monolayers were incubated at 37° C. with peptide solutions at various concentrations for 24 hr. For detection of fluorescein-labeled peptides, cells were rinsed once with PBS (pH=7.3) and visualized using a fluorescence microscope (Axiovert 135 at 320×).

Evaluation f Growth Inhibition. 3×10³ cells/well were plated on a 96 well plate at 10% FCS and cultured overnight. The culture medium was discarded, and the cells were washed once with Opti-MEM. The cell monolayers were incubated at 37° C. with peptide solutions at various concentrations for 24 hr. The growth inhibition was evaluated using solutions composed of 3-(4,5-dimethylthiazol-2-yl)-5-(3-carboxymethoxyphenyl)-2-(4sulfophenyl)-2H-tetrazolium; MTS (from Promega) and phenazine methosulfate; PMS (from Sigma). Concentrations inhibiting the growth by 50% (IC₅₀) were calculated after 24 hr.

Peptides. Tat-peptides are synthesized by solid-phase chemistry on an Applied Biosystems 433A peptide synthesizer. Peptides were labeled with fluorescein maleimide on their cysteine residue. Penetratin-peptides were synthesized using available techniques. The amino acid sequences of the peptides are in Table VII.

TABLE VII
Sequence of Various Peptides

| Name | Sequence |
|---|---|
| Tat | YGRKKRRQRRRG |
| Tat-linear | YGRKKRRQRRRG PVKRRLDL |
| Tat-cyclic | YGRKKRRQRRRG PAKR(KLFG) |
| Tat-Smt (scrambled) | YGRKKRRQRRRGRLDLPKVRKRS |
| Tat-Umt (unrelated) | YGRKKRRQRRRGETDHQYLAESS |
| FITC-Tat-mt | FluMalCXYGRKKRRQRRRG PVKARLDL |
| Penetratin | RQIKIWFQNRRMKWKK |
| Penetratin-linear | RQIKIWFQNRRMKWKKPVKRRLFG |

Where amino acids are represented by their universal single amino acid codes, Flu is fluorescein, Mal is maleimido and X is a Gly or Gly-Gly linker.

Results

Inhibition of E2F-1/Cyclin A Binding. As the E2F-1 derived eight-residue peptide (87-94) can disrupt the binding of Cyclin A-cdk2 complexes to E2F-1 and p21, introduction of these peptides into mammalian cells may provide a means to assess the physiologic consequences of inactivating the E2F-1/Cyclin A heterodimer. An internalization sequence derived from either HIV-tat 47-56 or 16 amino acid residues taken from the third helix of the Drosophila melanogaster Antennapedia homeodomain protein (Table VII) has been shown to translocate across biological membranes. Tat sequence was attached to either E2F-1 Pro-Val-Lys-Arg-Arg-Leu-Asp-Leu, cyclized consensus Pro-Ala-Lys-Arg-(Lys-Leu-Phe-Gly), or scrambled linear 8-mer to create Tat-linear, Tat-cyclized, or Tat-mt. Similar nomenclature was made for penetratin series (Table VII). The inhibitory activity of these fusion peptides on E2F-1/Cyclin A binding was determined. Tat-linear, Tat-cyclic and penetratin-linear showed 50% inhibition at range 0.1-1 μM on E2F-1/Cyclin A binding assay.

The IC₅₀ value is about 1-100 fold higher than those without fusion of nuclear localization sequences. In contrast, Tat, Tat-scrambled or penetratin peptides showed no inhibition up to 100 μM. Similar results were obtained from GST-pull down assay using in vitro translation assay.

Uptake and Intracellular Compartmentalization of Tat Peptides. The internalization of the Tat-mt peptide labeled with fluorescein maleimide on the cysteine residue was investigated. The labeled peptide was verified by mass spectrometry and purified by HPLC before use. When added to the cell culture optimum medium of MDA-MB-435 cells, the peptide was mainly recovered from the nucleus with a nucleolar accumulation after 30 min of incubation only. We further repeated the experiments with incubation of the peptide at 30 and 100 μM for 24 hr in osteosarcoma U2OS cells. The results showed 100% penetration to the nucleus. The peptide was tested under the same conditions after direct labeling with fluorescein maleimide. No variation in the amount and localization of the internalized peptide was observed as compared with the Tat-peptide.

Growth Inhibition. When an asynchronous culture of U2OS was treated with Tat-linear or Tat-cyclic, beginning at about 3 hr post-treatment and continuing thereafter, the cells adopted to a rounded morphology. They showed dose-dependent inhibition as measured by the MTS assay with more pronounced effect in the case of cyclic peptide. The effect was specific for the fusion peptide as cells treated with mer linear, cyclic (without the additional nuclear localization sequence) failed to show any morphological alterations. Similarly, introduction of Tat by itself or Tat fusion peptides with scrambled or unrelated sequence failed to cause any morphological changes and MTS reading up to 300 μM. Other cell types, such MDA-MB435, MDA-MB231 breast carcinoma cells, HCT-116, SW480 colon carcinoma cells, W138VA13 SV40 transformed lung fibroblast were also susceptible to both Tat-linear and cyclic. peptides, with the exception of Rat1 and HeCat cells. Similar inhibition effect was seen when penetratin-wt sequence was introduced to A549 lung carcinoma and other tumor cell types.

The IC₅₀ values of different peptides were calculated and are summarized in Table VIII. The Tat-cyclic peptide is more potent than Tat-linear in cells which is in agreement with their IC₅₀ values in vitro. Furthermore, the tumor cell lines was inhibited more than immortalized normal cell lines. Possibly, the endogenous basal level of E2F-1 in tumor cells are higher as previously revealed by immunoblotting.

TABLE VIII
IC₅₀ (μM) of Various Peptides

| Cell Lines | Tat-linear | Tat-Cyclic | Tat-mt | Penetratin-wt |
|---|---|---|---|---|
| U2OS | 28-40 | 6-7 | >100 | 7 |
| MDA-MB-435 | 48-50 | 6-7 | >100 | 18 |
| MDA-MB-231 | NT | 6-7 | NT | 8 |
| HCT-116 | 80 | NT | NT | NT |
| SW480 | 26 | NT | NT | NT |
| WI38/VA13 SV40 | NT | 6-7 | NT | 14 |
| A549 | NT | 26-46 | >100 | 22-25 |
| Rat1 | >100 | NT | NT | NT |
| HeCat | >100 | NT | NT | NT |

*NT, not tested

Inhibition of the E2F-1/Cyclin A/cdk2 Binding by Various Peptides

To evaluate the minimum sequence required for binding to Cyclin A, peptides of various lengths are made and tested for their inhibitory activity in the E2F-1/Cyclin A/cdk2 ELISA. The IC₅₀ values for the cyclic 8-mer and the cyclic 6-mer are 1 nM and 20 nM, respectively. However, the IC₅₀value for the cyclic 5-mer is 3 μM, or 2 orders of magnitude higher than the cyclic 8-mer. Thus, a 6 amino-acid peptide appears to be the minimum length for an active peptide. In addition, the cyclic peptides are more potent than the corresponding linear sequences.

ELISA

Nunc Immulon II ELISA plates are coated overnight at 4° C. with 250 μL of 4 mg/mL anti-GST antibody (Pharmacia Biotech) in bicarbonate buffer. Following five washes with wash buffer consisting of 50 mM Tris (pH=7.5), 0.15 M NaCI, and 0.01 % Tween-20 (TBST), non-specific sites were blocked for 2 hr at room temperature with 300 μL of assay buffer consisting of 50 mM HEPES (pH=7.5), 0.15 M NaCl, 0.1% Triton X-100, and 5% bovine serum albumin (BSA). Plates were then washed five times in TBST, aspirated dry, and treated with 100 μL of GST-E2F-1 in TBS (25 nM). GST-E2F-1 was incubated at RT for at least 1 hr with non-specific binding (NSB) control wells receiving assay buffer with no protein. Plates were washed five times in TBST and multiple concentrations of test compounds diluted in assay buffer were co-incubated with 5 nM Cyclin A/cdk2 diluted in assay buffer. The Cyclin A/cdk2 complex was freshly prepared by mixing a 1:1 ratio of the two proteins in TBS at 4° C. for 30 min prior to addition to the assay plate. Following incubation for 2 hr at RT, plates were washed five times in TBST, and 100 μL of a 1:500 dilution of a rabbit anti-cdk2 antibody (Santa Cruz) diluted in assay buffer and were added to all wells. Following incubation for at least 1 hr at RT or overnight at 4° C., the plates were washed five times in TBST and 100 μL of a 1:1000 dilution of a HRP-conjugated anti-rabbit IgG antibody (Pierce) were added to all wells. Following a half hour incubation at RT, the plates were washed five times in TBST, aspirated dry, and developed by the addition of 100 μL of a HRP substrate ABTS prepared in sodium citrate buffer (pH=4.2). After 10-15 min, absorbance is read using a microplate reader at 405 nm.

While the invention has been described and illustrated with reference to certain particular embodiments thereof, those skilled in the art will appreciate that various changes, modifications and substitutions can be made therein without departing from the spirit and scope of the invention. For example, effective dosages other than the particular dosages as set forth herein above may be applicable as a consequence of variations in the responsiveness of the mammal being treated for any of the indications for the compounds of the invention indicated above. Likewise, the specific pharmacological responses observed may vary according to and depending upon the particular active compound selected or whether there are present pharmaceutical carriers, as well as the type of formulation and mode of administration employed, and such expected variations or differences in the results are contemplated in accordance with the objects and practices of the present invention. It is intended, therefore, that the invention be defined by the scope of the claims which follow and that such claims be interpreted as broadly as is reasonable.

### EXAMPLE 1

### Synthesis of the cyclic 6-mer: Cpr-Lys-(CH(CH₃)(C₁₃H₂₇))-Lys-(Lys-Leu-Phe-Gly)

The synthesis of the cyclic 6-mer utilizes the commercially available Fmoc-Gly-SASRIN resin as a starting point. The chain of the 6-mer is elaborated in the ‘C’ to ‘N’ direction by sequential deprotection with 25% piperidine followed by HBTU-mediated coupling with Fmoc-L-Phe-OH, Fmoc-L-Leu-OH, Fmoc-L-Lys(Mtt)-OH, Fmoc-L-Lys(Boc)-OH, Fmoc-L-Lys(Dde)-OH, and finally with cyclopropanecarboxylic acid. Once the peptide is assembled on the solid-phase on an Applied Biosystems ABI433A peptide synthesizer, protected peptide-resin (2 mM) is treated with 30 mL of 2% NH₂NH₂ in DMF twice (30 min and 90 min). The resin is washed well with DMF (2×), CH₂Cl₂ (2×), MeOH (1×), and CH₂Cl₂. The peptide resin is reductively alkylated at Lys-1 with heptadecanone and NaBH₃CN (0.75 g) in THF/MeOH (50 mL, 1:1) and catalytic amount of AcOH (3 drops). The Mtt group of Lys4 is selectively removed by 1% TFA in CH₂Cl₂ (3 times, 60 mL each). These conditions also cleave the protected peptide from the resin and the peptide was concentrated. The crude protected linear peptide is then cyclized between the side-chain amino group of Lys-4 and α-carboxyl group of Gly using HBTU (11.25 mM)/HOBt (11.25 mM)/DIEA (12 mL) DMF (25 mL) for 30 min. The cyclic peptide is precipitated in cold water (2 L) and filtered. The cyclic peptide is deprotected with 50% TFA/H₂O (100 mL) for 2 hr to provide crude peptide which was precipitated in cold diethyl ether. The crude product (1.2 g) is purified by reverse-phase HPLC using C8 column and a gradient of CH₃CN (+0.1% TFA) in H₂0(+0.1% TFA). Fractions containing homogeneous material are pooled and lyophilized to a white flocculent powder. The pure 6-mer peptide exhibits m/z (MH+) 1008.7 and is obtained in 24 % (480 mg) overall yield.

### EXAMPLE 2

### Synthesis of the cyclic 8-mer, Pro-Ala-Lys-Arg-(Lys-Leu-Phe-Gly)

The synthetic procedure for the cyclic 8-mer, Pro-Ala-Lys-Arg-(Lys-Leu-Phe-Gly) is described. The synthesis of the cyclic 8-mer, ProAlaLysArg(LysLeuPheGly), utilizes the commercially available Fmoc-Gly-SASRIN resin as a starting point. The chain of the cyclic 8mer, Pro-Ala-Lys-Arg-(Lys-Leu-Phe-Gly), is elaborated in the ‘C’ to ‘N’ direction by sequential deprotection with 25% piperidine followed by HBTU-mediated coupling with Fmoc-L-Phe-OH, Fmoc-L-Leu-OH, Fmoc-L-Lys(Mtt)-OH, Fmoc-L-Arg(Pbf)-OH, Fmoc-L-Lys (t-Boc)-OH, Fmoc-L-Ala-OH, and finally with Boc-L-Pro-OH. Once the peptide is assembled on the solid-phase, the Mtt group of Lys-5 is selectively removed by 1% TFA in CH₂Cl₂. These conditions also cleave the protected peptide from the resin. The crude protected linear peptide (FAB-MS, m/z 1168) is then cyclized between the side-chain amino group of Lys-5 and the α-carboxyl group of Gly-8 using HBTU/HOBt/DMF. The cyclic peptide is deprotected with 95% TFA/H₂O for 1 hr to provide crude peptide cyclic 8-mer, Pro-Ala-Lys-Arg-(Lys-Leu-Phe-Gly). The crude product is purified by reverse-phase HPLC using a gradient of CH₃CN (+0.1% TFA) in H₂O (+0.1% TFA). Fractions containing homogeneous material are pooled and lyophilized to a white flocculent powder. The clean cyclic 8-mer peptide, Pro-Ala-Lys-Arg-(Lys-Leu-Phe-Gly), exhibits m/z (MH+) 899.13 consistent with the calculated molecular weight of the cyclic 8-mer, Pro-Ala-Lys-Arg-(Lys-Leu-Phe-Gly), FAB-MS, MH+ for C₄₃H₇₁N₁₃O₈, Structure of the cyclic 8-mer, Pro-Ala-Lys-Arg-(Lys-Leu-Phe-Gly):

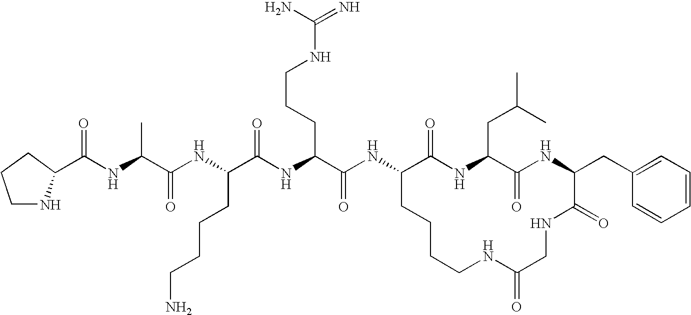

What is claimed is:

1. An isolated peptide comprising the amino acid sequence selected from the general structural formula Ia, Ib, Ic and Id:

| | | |
|---|---|---|
| Cap-AA8-AA7-AA6-AA5-AA4*-AA3-AA2-AA1* | 8-mer | la |
| Cap-AA7-AA6-AA5-AA4*-AA3-AA2-AA1* | 7-mer | lb |
| Cap-AA6-AA5-AA4*-AA3-AA2-AA1* | 6-mer | lc |
| Cap-AA5-AA4*-AA3-AA2-AA1* | 5-mer | ld |

wherein
AA1 is selected from:
(a) Gly,
(b) Ala,
(c) Leu, and
(d) a small aliphatic amino acid;
AA2 is selected from:
(a) Phe,
(b) Tha,
(c) Cha,
(d) Tyr,
(e) Pya,
(f) Trp, and
(g) another aromatic amino acid;
AA3 is selected from:
(a) Leu,
(b) Cpa, and
(c) a natural or unnatural aliphatic amino acid;
AA4 is selected from:
(a) Lys,
(b) Lys substituted by C₁-C₁₇ alkyl, C₅-C₂₀ arylalkyl or a C₆-C₂₀ aryl radical,
(c) Orn optionally substituted by C₁-C₁₇ alkyl, C₅-C₂₀ arylalkyl or a C₆-C₂₀ aryl radical, and
(d) hLys optionally substituted by C₁-C₁₇ alkyl, C₅-C₂₀ arylalkyl or a C₆-C₂₀ aryl radical;
AA5 is selected from:
(a) Arg,
(b) Lys,
(c) Orn,
(d) hLys,
(e) His, and
(f) Lys wherein N^{ε} is substituted by one or two radicals selected from C₅-C₂₀ alkyl, a linear or branched C₁-C₆ acyl group, cyclized saturated or unsaturated C₅-C₂₀ alkyl, C₅-C₂₀ arylalkyl and a C₆-C₂₀ aryl radical;
AA6 is selected from:
(a) Lys,
(b) hLys,
(c) Orn,
(d) Lys wherein N^{ε} is substituted by one or two radicals selected from C₅-C₂₀ alkyl, a linear or branched C₁-C₆ acyl group, cyclized saturated or unsaturated C₅-C₂₀ alkyl, C₅-C₂₀ arylalkyl and a C₆-C₂₀ aryl radical, and
(e) Orn wherein N^{δ} is substituted by one or two radicals selected from C₅-C₂₀ alkyl, a linear or branched C₁-C₆ acyl group, cyclized saturated or unsaturated C₅-C₂₀ alkyl, C₅-C₂₀ arylalkyl and a C₆-C₂₀ aryl radical, and
(f) Pro;
AA7 is selected from:
(a) Ala,
(b) Val, and
(c) a natural or unnatural amino acid, or mimetics or isostere thereof;
AA8 is selected from:
(a) Pro,
(b) a natural or unnatural amino acid, or mimetics or isostere thereof; and
the Cap is either not present or selected from:
(a) C₁-C₈ acyl, and
(b) C₃-C₈ cycloalkylalkanoyl or furanylacetyl;
and pharmaceutically acceptable salts thereof;
such a peptide being optionally linked to nuclear localization peptide sequences HIV-1 Tat or Antennapedia peptide sequence (penetratin);
and the (*) symbol indicates a site for optional intramolecular linkage via an amide, substituted amide bond or isostere thereof; the resulting compounds being the respective cyclic 5-mers, 6-mers, 7-mers, or 8-mers.

2. An isolated peptide according to claim 1, wherein
AA1 is selected from:
(a) Gly,
(b) Ala, and
(c) Leu;
AA2 is selected from:
(a) Phe,
(b) Tha,
(c) Cha,
(d) Tyr,
(e) Pya, and
(f) Trp;
AA3 is selected from:
(a) Leu,
(b) Cpa, and
(c) a natural aliphatic amino acid;
AA4 is selected from:
(a) Lys,
(b) Orn, and
(c) hLys;
AA5 is selected from:
(a) Arg,
(b) Lys,
(c) Orn,
(d) hLys, and
(e) His;
AA6 is selected from:
(a) Lys,
(b) hLys,
(c) Orn;
AA7 is selected from:
(a) Ala,
(b) Val, and
(c) a natural amino acid;
AA8 is selected from:
(a) Pro,
(b) a natural amino acid; and
the Cap is either not present or selected from:
(a) acetyl (Ac), cyclopropylcarbonyl, cyclopropylacetyl (Cpr), pivaloyl, isopropylcarbonyl, isopropylacetyl, 2,2-dimethylbutanoyl (Dmb), levulinoyl, cyclopropylglycinoyl (Cpg), dimethylglycinoyl (Dmg), and
(b) cyclopentylacetyl, cyclohexylacetyl, cycloheptylacetyl, furanylacetyl;
and pharmaceutically acceptable salts thereof;
such a peptide being optionally linked to nuclear localization peptide sequences HIV-1 Tat or Antennapedia peptide sequence (penetratin);
and the (*) symbol indicates a site for optional intramolecular linkage via an amide bond; the resulting compounds being the respective cyclic 5-mers, 6-mers, 7-mers, or 8-mers.

3. An isolated peptide according to claim 1 comprising:
the cyclic 5-mer:
Ac-Arg-(Lys-Leu-Phe-Gly), or
Ac-Lys-(Lys-Leu-Phe-Gly);
the cyclic 6-mer:
Ac-Lys-Arg-(Lys-Leu-Phe-Gly),
Ac-Lys-Lys-(Lys-Leu-Phe-Gly),
Cpr-Lys-Arg-(Lys-Leu-Phe-Gly),
Cpr-Lys-Lys-(Lys-Leu-Phe-Gly),
Cpr-Lys-(C₅-C₂₀)-Lys-(Lys-Leu-Phe-Gly),
Cpr-Lys-(C₅-C₂₀)-Arg-(Lys-Leu-Phe-Gly),
Cpr-Lys-(CH(CH₃)(C₁₃H₂₇))-Lys-(Lys-Leu-Phe-Gly),
Dmb-Lys-(C₅-C₂₀)-Arg-(Lys-Leu-Phe-Gly), or
Dmb-Lys-(C₅-C₂₀)-Lys-(Lys-Leu-Phe-Gly);
the cyclic 7-mer:
Ac-Ala-Lys-Arg-(Lys-Leu-Phe-Gly),
Ac-Ala-Lys-Lys-(Lys-Leu-Phe-Gly),
Cpr-Ala-Lys-Arg-(Lys-Leu-Phe-Gly), or
Cpr-Ala-Lys-Lys-(Lys-Leu-Phe-Gly); or
the cyclic 8-mer:
Pro-Ala-Lys-Arg-(Lys-Leu-Phe-Gly),
Ac-Pro-Ala-Lys-Arg-(Lys-Leu-Phe-Gly),
Ac-Pro-Ala-Lys-Lys-(Lys-Leu-Phe-Gly),
Cpr-Pro-Ala-Lys-Arg-(Lys-Leu-Phe-Gly), or
Cpr-Pro-Ala-Lys-Lys-(Lys-Leu-Phe-Gly);
wherein parentheses indicate the residues involved in cyclization;
and pharmaceutically acceptable salts of such peptides.

4. A peptide according to claim 1 or a pharmaceutically acceptable salt thereof for use in a method for the therapeutic treatment of a mammal.

5. A pharmaceutical composition comprising a peptide according to claim 1, or a pharmaceutically acceptable salt thereof, together with a pharmaceutically acceptable carrier.

6. A pharmaceutical composition for the treatment of cancer in a mammal comprising, in a therapeutically effective amount, a peptide according to claim 1, or a pharmaceutically acceptable salt thereof, together with a pharmaceutically acceptable carrier.

7. The use of a peptide according to claim 1 or a pharmaceutically acceptable salt thereof for the preparation of a pharmaceutical composition for use in the treatment of cancer.

8. The use of a peptide according to claim 1 or a pharmaceutically acceptable salt thereof in the treatment of cancer.

9. A method for treating cancer comprising administering to a mammal in need of such treatment a therapeutically effective amount of a peptide according to claim 1, or a pharmaceutically acceptable salt thereof.

10. A method of inhibiting the binding of the E2F-1 cell regulatory protein to Cyclin A comprising administering to a mammal in need of such treatment a therapeutically effective amount of a peptide according to claim 1, or a pharmaceutically acceptable salt thereof.
